(19) United States
(12) Patent Application Publication
(10) Pub. No.: US 2026/0013836 A1
(43) Pub. Date: Jan. 15, 2026

(54) ULTRASONIC DETECTION CIRCUIT AND DRIVING METHOD THEREFOR, AND ULTRASONIC DETECTION DEVICE

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Liwei Liu, Beijing (CN); Yue Tong, Beijing (CN); Zhuo Li, Beijing (CN); Xuan Feng, Beijing (CN); Hui Zhang, Beijing (CN); Seungwoo Han, Beijing (CN); Ming Yang, Beijing (CN); Dingchang Zhang, Beijing (CN); Yonggang Cao, Beijing (CN)

(73) Assignees: BEIJING BOE TECHNOLOGY DEVELOPMENT CO., LTD., Beijing (CN); BOE TECHNOLOGY GROUP CO., LTD., Beijing (CN)

(21) Appl. No.: 18/992,789

(22) PCT Filed: Apr. 18, 2024
(86) PCT No.: PCT/CN2024/088661

§ 371 Date: Jan. 9, 2025

(30) Foreign Application Priority Data
May 26, 2023 (CN) 202310611551.0

(51) Int. Cl. A61B 8/00; B06B 1/02

(52) U.S. Cl. CPC A61B 8/54; A61B 8/4411; B06B 1/0207; B06B 2201/76

(57) ABSTRACT

An ultrasonic detection circuit includes a reset module, an acquisition module, a drive transistor, a storage module, a compensation module and a reading module; wherein the reset module is respectively connected to a reset control end, a reset signal end and a first node; the acquisition module is connected to an acquisition control end, an ultrasonic sensor and the first node; the drive transistor and the control electrode are connected to the first node, the first electrode is connected to the compensation module, and the second electrode is connected to the second node; the storage module is respectively connected to the first node and the second node; the compensation module is also connected to a compensation control end and a first voltage end; and the reading module is respectively connected to a read control end, the second node and an output end.

### CROSS-REFERENCE TO RELATED APPLICATIONS

The present disclosure claims priority to Chinese Patent Application No. 202310611551.0 filed on May 26, 2023 to the CNIPA, entitled “Ultrasonic detection circuit and driving method therefor, and ultrasonic detection device”, the entire contents of which are incorporated herein by reference.

### TECHNICAL FIELD

The present disclosure relates to the technical field of ultrasonic detection, and in particular, to an ultrasonic detection circuit, a driving method thereof, and an ultrasonic detection device.

### BACKGROUND

Ultrasonic imaging is an important non-destructive testing method in medical treatment. With the development of technology, the ultrasonic imaging is developing towards a faster, clearer and three-dimensional direction. High definition and high resolution are goals that have been explored and pursued to facilitate earlier discovery of diseases for timely treatment.

### SUMMARY

The present disclosure provides an ultrasonic detection circuit including a reset module, an acquisition module, a drive transistor, a storage module, a compensation module and a reading module;
wherein the reset module is respectively connected to a reset control end, a reset signal end and a first node, and is configured to write a reset signal of the reset signal end into the first node in response to a reset control signal of the reset control end;
the acquisition module is connected to an acquisition control end, an ultrasonic sensor and the first node, and is configured to write a signal received by the ultrasonic sensor into the first node in response to an acquisition control signal of the acquisition control end;
the drive transistor and a control electrode are connected to the first node, a first electrode is connected to the compensation module, and a second electrode is connected to the second node;
the storage module is respectively connected to the first node and the second node, and is configured to store a voltage between the first node and the second node;
the compensation module is further connected to a compensation control end and a first voltage end, and is configured to write a threshold voltage of the drive transistor into the second node in response to a compensation control signal of the compensation control end; and
the reading module is respectively connected to a read control end, the second node and an output end, and is configured to output a detection signal at the output end according to potentials of the first node and the second node in response to a reading control signal of the read control end.

In some embodiments, the reset module is directly connected to the first node; or
the reset module is connected to the first node via the acquisition module, and the reset module is specifically configured to write the reset signal into the acquisition module in response to the reset control signal; and the acquisition module is also configured to write the reset signal into the first node in response to the acquisition control signal.

In some embodiments, the reset module includes:
a second transistor, wherein a control electrode is connected to the reset control end, a first electrode is connected to the reset signal end, and a second electrode is connected to the first node directly or via the acquisition module.

In some embodiments, the acquisition module includes:
a third transistor, wherein a control electrode is connected to the acquisition control end, a first electrode is connected to the ultrasonic sensor, or the first electrode is respectively connected to the ultrasonic sensor and the reset module, and a second electrode is connected to the first node.

In some embodiments, the storage module includes:
a first capacitor, wherein a first electrode is connected to the first node, and a second electrode is connected to the second node.

In some embodiments, the compensation module includes:
a fourth transistor, wherein a control electrode is connected to the compensation control end, a first electrode is connected to the first voltage end, and a second electrode is connected to a first electrode of the drive transistor.

In some embodiments, the reading module includes:
a fifth transistor, wherein a control electrode is connected to the read control end, a first electrode is connected to the second node, and a second electrode is connected to the output end.

In some embodiments, the ultrasonic detection circuit further includes:
a control signal generation module respectively connected to the reset control end, the read control end and the compensation control end and configured to generate the compensation control signal at the compensation control end in response to the reset control signal and the read control signal.

In some embodiments, the control signal generation module includes:
a sixth transistor, wherein a control electrode and a first electrode are all connected to the reset control end, and a second electrode is connected to the compensation control end; and
a seventh transistor, wherein a control electrode and a first electrode are all connected to the read control end, and a second electrode is connected to the compensation control end.

The present disclosure provides an ultrasonic detection device, including:
an underlayer substrate;
a plurality of detection units located on a side of the underlayer substrate, wherein the detection units includes an ultrasonic sensor, and the ultrasonic detection circuit according to any one of the embodiments, and the ultrasonic sensor is configured to receive an ultrasonic signal reflected back and convert the received ultrasonic signal into an electrical signal.

In some embodiments, the plurality of detection units are arrayed in a row direction and/or a column direction.

In some embodiments, the ultrasonic sensor is further configured to emit an ultrasonic signal in response to a first driving signal; or
the ultrasonic detection device further includes an ultrasonic emitter, and the ultrasonic emitter is configured to emit an ultrasonic signal in response to a second driving signal.

The present disclosure provides a driving method applied to the ultrasonic detection circuit according to any one of the embodiments, wherein the driving method includes:
a reset compensation stage, providing a reset control signal to the reset control end and providing a compensation control signal to the compensation control end, so that the reset module and the compensation module are conductive, writing the reset signal into the first node, and writing the threshold voltage of the drive transistor into the second node;
an acquisition stage, providing an acquisition control signal to the acquisition control end to enable the acquisition module to conduct, and writing a signal received by the ultrasonic sensor into the first node; and
a reading stage, providing a compensation control signal to the compensation control end and providing a reading control signal to the read control end, so that the compensation module and the reading module are conductive, and outputting a detection signal at the output end according to the potentials of the first node and the second node.

In some embodiments, when the reset module is connected to the first node by the acquisition module, the reset compensation stage further includes:
providing an acquisition control signal to the acquisition control end to conduct the acquisition module, wherein the reset signal is written into the first node via the reset module and the acquisition module in sequence.

In some embodiments, when the ultrasonic detection circuit further includes a control signal generation module, and the control signal generation module is respectively connected to the reset control end, the read control end and the compensation control end, a step of providing a compensation control signal to the compensation control end includes:
providing a reset control signal to the reset control end and providing a read control signal to the read control end to generate the compensation control signal at the compensation control end.

In some embodiments, after a previous acquisition stage and before a next acquisition stage, the driving method further includes:
an emitting stage, providing a first driving signal to the ultrasonic sensor to cause the ultrasonic sensor to emit an ultrasonic signal.

The above description is only an overview of the disclosed technical solution. In order to have a clearer understanding of the disclosed technical solution, it can be implemented according to the content of the specification. In order to make the above and other purposes, features, and advantages of the present disclosure more obvious and understandable, the specific implementation methods of the present disclosure are listed below.

### BRIEF DESCRIPTION OF THE DRAWINGS

In order to provide a clearer explanation of the technical solutions in the disclosed embodiments or related art, a brief introduction will be given to the appended drawings required for the description of the embodiments or related art. It is obvious that the appended drawings in the following description are some embodiments of the present disclosure. For those skilled in the art, other drawings can be obtained based on these drawings without expenditure of creative labor. It should be noted that the scale in the appended drawings is only for illustration and does not represent the actual scale.

FIG. 1 exemplarily shows a structurally schematic view of an ultrasonic detection circuit in the related art;

FIG. 2 exemplarily shows a structurally schematic view of an ultrasonic detection circuit provided by the present disclosure;

FIG. 3 exemplarily shows a structurally schematic view of a second ultrasonic detection circuit provided by the present disclosure;

FIG. 4 exemplarily shows an equivalent circuit diagram of a first ultrasonic detection circuit at various stages;

FIG. 5 exemplarily shows an equivalent circuit diagram of a second ultrasonic detection circuit at various stages;

FIG. 6 exemplarily shows a structurally schematic view of a third ultrasonic detection circuit provided by the present disclosure;

FIG. 7 exemplarily shows a structurally schematic view of a fourth ultrasonic detection circuit provided by the present disclosure;

FIG. 8 exemplarily shows a timing chart of a driving signal of a first ultrasonic detection circuit; and

FIG. 9 exemplarily shows a timing diagram of a driving signal of a second ultrasonic detection circuit.

### DETAILED DESCRIPTION OF THE EMBODIMENTS

In order to clarify the purpose, technical solution, and advantages of these disclosed embodiments, a clear and complete description of the technical solution in these disclosed embodiments will be provided below in conjunction with the appended drawings. Obviously, the described embodiments are a part of these disclosed embodiments, not the entire embodiment. Based on the embodiments disclosed in the present disclosure, all other embodiments obtained by persons skilled in the art without expenditure of creative labor are within the scope of protection of the present disclosure.

The traditional ultrasonic imaging technology starts from the characteristics of waveform time distribution, signals from time to time, uses full-wave imaging algorithm, and mostly adopts one-dimensional array element design. The recognition resolution can reach 1 mm. Based on the spatial distribution characteristics of the wave front, the large-area two-dimensional array element design can improve the resolution, and the recognition resolution can reach 0.5 mm-1 mm.

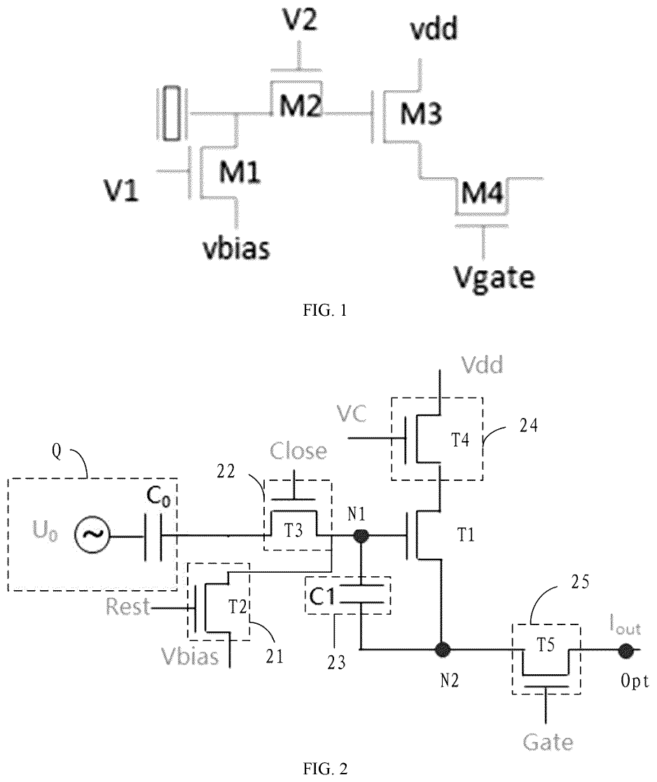

In the related art, the ultrasonic detection circuit in an array element uses a 4T circuit as shown in FIG. 1. Since the performance uniformity of a thin film transistor in a large area range is poor, for example, the threshold voltage difference of M3 transistors in different positions is large, collected signal is distorted finally.

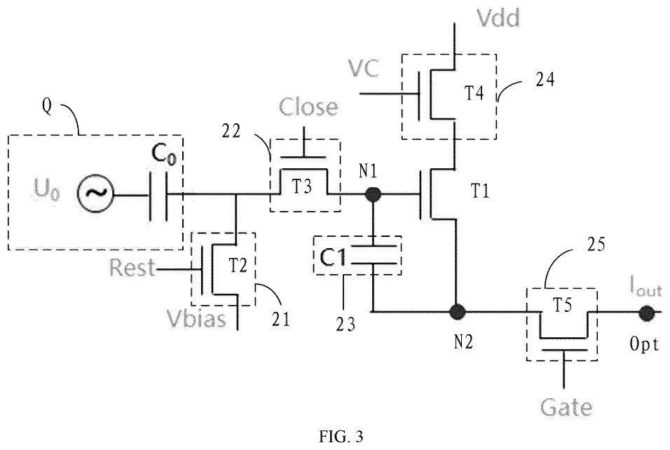

In order to solve the above-mentioned problem, a schematic structural diagram of an ultrasonic detection circuit provided by the present disclosure is exemplarily shown with reference to FIG. 2 and FIG. 3, respectively. As shown in FIG. 2 or 3, the ultrasonic detection circuit includes a reset module 21, an acquisition module 22, a drive transistor T1, a storage module 23, a compensation module 24 and a reading module 25.

The reset module 21 is respectively connected to the reset control end Rest, the reset signal end Vbias and a first node N1, and the reset module 21 is configured to write the reset signal of the reset signal end Vbias into the first node N1 in response to the reset control signal of the reset control end Rest.

The acquisition module 22 is connected to the acquisition control end Close, the ultrasonic sensor Q and the first node N1, and the acquisition module 22 is configured to write a signal received by the ultrasonic sensor Q into the first node N1 in response to an acquisition control signal of the acquisition control end Close.

A control electrode of the drive transistor T1 is connected to the first node N1, a first electrode of the drive transistor T1 is connected to the compensation module 24, and a second electrode of the drive transistor T1 is connected to the second node N2.

The storage module 23 is connected to the first node N1 and the second node N2, respectively, and the storage module 23 is configured to store the voltage between the first node N1 and the second node N2.

The compensation module 24 is further connected to a compensation control end VC and a first voltage end Vdd. The compensation module 24 is adapted to write the threshold voltage Vth of the drive transistor T1 to the second node N2 in response to a compensation control signal at the compensation control end VC.

The reading module 25 is respectively connected to a read control end Gate, a second node N2 and an output end Opt. The reading module 25 is configured to output a detection signal at the output end Opt according to the potentials of the first node N1 and the second node N2 in response to a reading control signal of the read control end Gate.

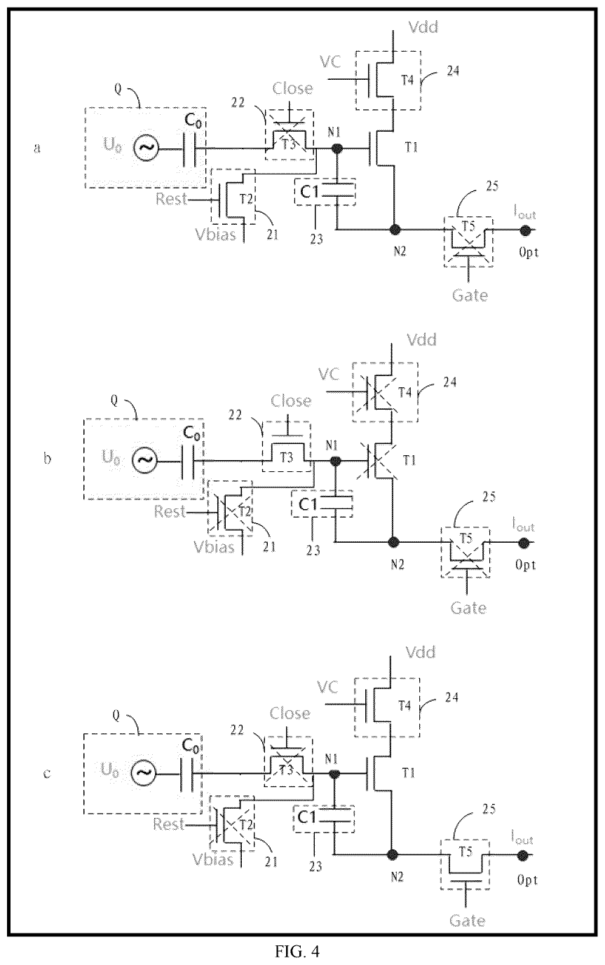

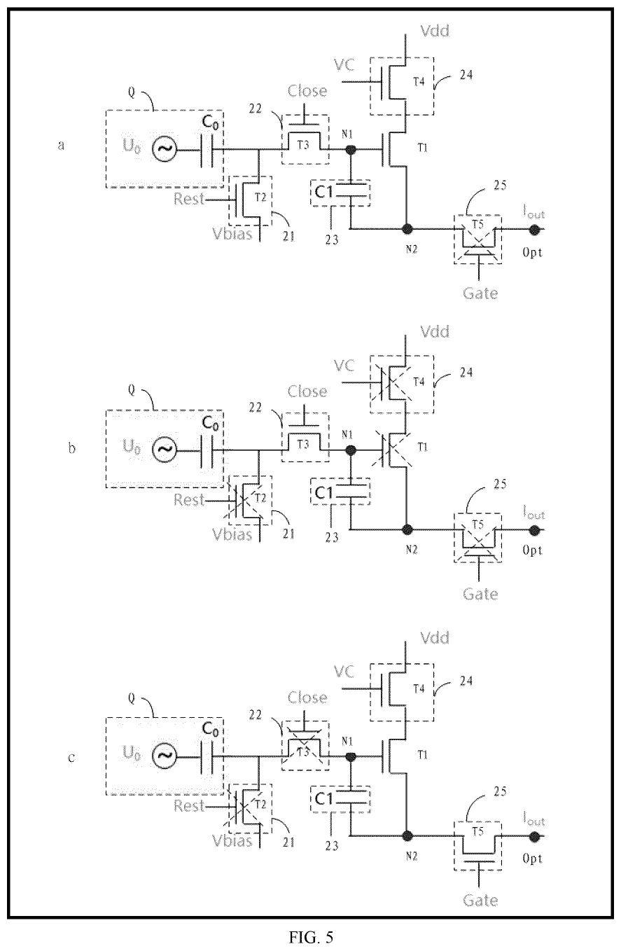

First, a reset control signal can be provided to the reset control end Rest and a compensation control signal can be provided to the compensation control end VC to conduct the reset module 21 as well as the compensation module 24 during a reset compensation stage t1. The reset signal is written to the first node N1 and the threshold voltage Vth of the drive transistor T1 is written to the second node N2, and an equivalent circuit of the ultrasonic detection circuit shown in FIG. 2 during this stage is shown with reference to a diagram in FIG. 4. After completion of the reset compensation stage t1, the voltage at the first node N1 is V1=Vbias and the voltage at the second node N2 is V2=Vbias−Vth.

Then, in an acquisition stage t2, an acquisition control signal may be supplied to the acquisition control end Close to conduct the acquisition module 22 and a signal received by the ultrasonic sensor Q is written to the first node N1. An equivalent circuit of the ultrasonic detection circuit shown in FIG. 2 in this stage is shown with reference to Panel b in FIG. 4, and an equivalent circuit of the ultrasonic detection circuit shown in FIG. 3 in this stage is shown with reference to Panel b in FIG. 5.

Thereafter, a compensation control signal may be supplied to the compensation control end VC and a read control signal may be supplied to the read control end Gate to conduct the compensation module 24 and the reading module 25 during the read stage t3. A detection signal may be output at the output end Opt according to the potentials of the first node N1 and the second node N2. An equivalent circuit of the ultrasonic detection circuit of FIG. 2 in this stage is shown with reference to Panel c in FIG. 4, and an equivalent circuit of the ultrasonic detection circuit of FIG. 3 in this stage is shown with reference to Panel c in FIG. 5. In the read stage t3, the saturation current on the drive transistor T1 can be expressed as:

$$Ids = 1 / 2 * Cox * u * W / L * {( Vgs - Vth )}^{∧} 2$$

Wherein, ½*Cox*u*W/L is a constant value, the saturation current is related to the gate-source voltage difference Vgs and the threshold voltage Vth. If the difference of the threshold voltage Vth of the drive transistor T1 in different positions is large, it may lead to a large difference of the current output by the output end Opt in different positions, thus leading to a large ultrasonic detection error.

In the present disclosure, the gate-source voltage difference Vgs is the voltage difference between the first node N1 and the second node N2, that is, Vgs=V1−V2. Since the second node N2 is written into the threshold voltage Vth of the drive transistor T1 during the reset compensation stage T1, and since the voltage difference between the first node N1 and the second node N2 always contains the threshold voltage Vth of the drive transistor T1 during the subsequent acquisition stage t2 and the read stage t3 due to the bootstrap effect of the storage module 23, Vth can be eliminated by substituting into the Ids formula, finally making the saturation current Ids only related to Vbias and the signal U0 received by the ultrasonic sensor Q. Herein, the Vbias can be at a constant level, so that the saturation current is only related to the signal U0 received by the ultrasonic sensor Q and is independent of the threshold voltage Vth of the drive transistor T1, thereby eliminating the influence of the difference in the threshold voltage Vth of the drive transistor T1 at different positions on the output current, reducing the error in ultrasonic signal detection and improving the detection accuracy.

It should be noted that in the read stage t3, since the compensation block 24, the drive transistor T1 and the read block 25 are connected in series with each other, the saturation current of the drive transistor T1, i.e., the read current of the output end Opt, can be considered.

The ultrasonic detection circuitry provided by the present disclosure may be applied to ultrasonic imaging to facilitate high resolution, and high precision ultrasonic imaging medical devices. For example, it can be applied to large area two-dimensional array element ultrasonic imaging device, which is beneficial to improve the resolution and accuracy of identification.

The ultrasonic detection circuit provided by the present disclosure may also be applied to fingerprint recognition, advantageously improving the accuracy of fingerprint recognition.

In some embodiments, as shown in FIG. 2, the reset module 21 is directly connected to the first node N1.

To prevent the first node N1 from leaking electricity, in some embodiments, as shown in FIG. 3, the reset module 21 is connected to the first node N1 by the acquisition module 22, and the reset module 21 is connected to the same end of the acquisition module 22 as the ultrasonic sensor Q. The reset module 21 is specifically configured to write the reset signal into the acquisition module 22 in response to the reset control signal, and the acquisition module 22 is also configured to write the reset signal into the first node N1 in response to the acquisition control signal.

In a specific implementation, in a reset compensation stage t1, an acquisition control signal may also be provided to an acquisition control end Close to conduct an acquisition module 22, and a reset signal is written to the first node N1 via the reset module 21 and the acquisition module 22 in sequence. An equivalent circuit of the ultrasonic detection circuit shown in FIG. 3 in this stage is shown with reference to Panel a in FIG. 5.

In some embodiments, as shown in FIG. 2 or FIG. 3, the reset module 21 includes a second transistor T2, a control electrode of the second transistor T2 is connected to a reset control end Rest, a first electrode of the second transistor T2 is connected to a reset signal end Vbias, a second electrode of the second transistor T2 is directly connected to the first node N1 (as shown in FIG. 2), or a second electrode of the second transistor T2 is connected to the first node N1 via an acquisition module 22 (as shown in FIG. 3). In FIG. 3, the second electrode of the second transistor T2 is connected to the same end of the acquisition module 22 as the ultrasonic sensor Q.

In some embodiments, as shown in FIG. 2 or FIG. 3, the acquisition module 22 includes a third transistor T3, a control electrode of the third transistor T3 is connected to the acquisition control end Close, a first electrode of the third transistor T3 is connected to the ultrasonic sensor Q (as shown in FIG. 2), or a first electrode of the third transistor T3 is connected to the reset module 21 and the ultrasonic sensor Q (as shown in FIG. 3). A second electrode of the third transistor T3 is connected to the first node N1.

In some embodiments, as shown in FIG. 2 or FIG. 3, the storage module 23 includes a first capacitor C1, a first electrode of which is connected to a first node N1, and a second electrode of which is connected to a second node N2.

In some embodiments, as shown in FIG. 2 or FIG. 3, the compensation module 24 includes a fourth transistor T4, a control electrode of the fourth transistor T4 is connected to the compensation control end VC, a first electrode of the fourth transistor T4 is connected to the first voltage end Vdd, and a second electrode of the fourth transistor T4 is connected to the first electrode of the drive transistor T1.

In some embodiments, as shown in FIG. 2 or FIG. 3, the reading module 25 includes a fifth transistor T5. A control electrode of the fifth transistor T5 is connected to the read control end Gate, a first electrode of the fifth transistor T5 is connected to the second node N2, and a second electrode of the fifth transistor T5 is connected to the output end Opt.

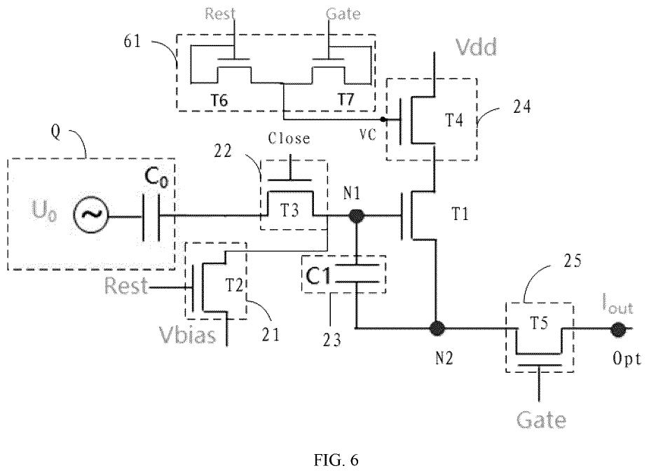

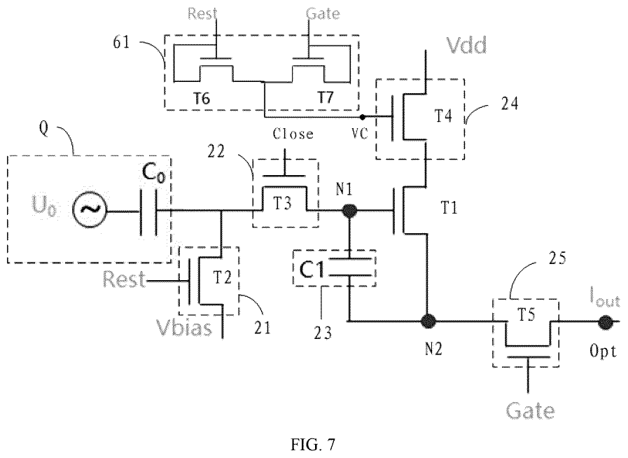

In some embodiments, as shown in FIG. 6 or FIG. 7, the ultrasonic detection circuit further includes a control signal generation module 61. The control signal generation module 61 is respectively connected to a reset control end Rest, a read control end Gate and a compensation control end VC. The control signal generation module 61 is configured to generate a compensation control signal at the compensation control end VC in response to the reset control signal and the read control signal.

By providing the control signal generation module 61, the compensation control signal is generated by multiplexing the reset control signal of the reset control end Rest and the read control signal of the read control end Gate, so that there is no need to provide a signal line for transmitting the compensation control signal, the quantity of signal lines is reduced, the wiring space is saved and the cost is reduced.

In some embodiments, as shown in FIG. 6 or FIG. 7, the control signal generation module 61 includes a sixth transistor T6 and a seventh transistor T7, wherein a control electrode and a first electrode of the sixth transistor T6 are both connected to the reset control end Rest, a second electrode of the sixth transistor T6 is connected to the compensation control end VC, a control electrode and a first electrode of the seventh transistor T7 are both connected to the read control end Gate, and a second electrode of the seventh transistor T7 is connected to the compensation control end VC.

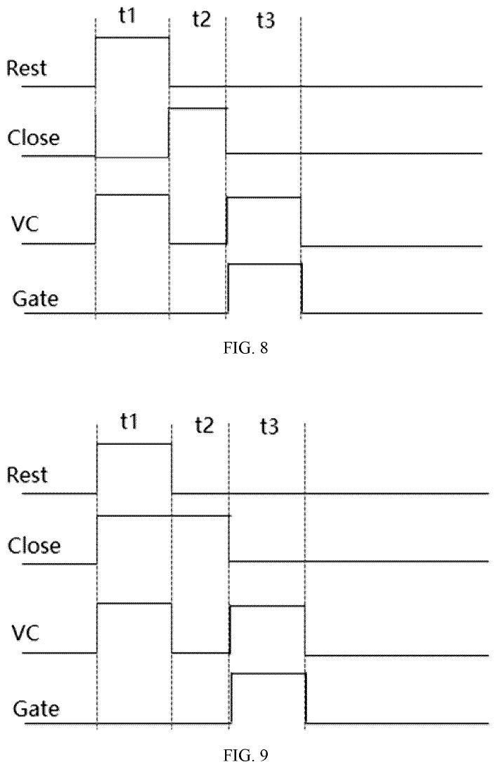

The operation of the ultrasonic detection circuit will now be described with reference to the signal timing diagrams shown in FIG. 2 and FIG. 8, taking the case where the drive transistors T1 to the fifth transistor T5 are all N-type transistors.

In the reset compensation stage t1, the reset control signal Rest and the compensation control signal VC are at a high level, and the acquisition control signal Close and the reading control signal Gate are at a low level. Therefore, the third transistor T3 and the fifth transistor T5 are turned off, the drive transistor T1, the second transistor T2 and the fourth transistor T4 are turned on, and the reset signal Vbias is charged into the first node N1, so that the voltage of the first node N1 is Vbias. After the voltage of the second node N2 reaches Vbias−Vth, the drive transistor T1 is turned off, thereby writing the threshold voltage Vth of the drive transistor T1 to the second node N2, which serves to compensate for the difference in the threshold voltage Vth during the read stage t3. The equivalent circuit of the ultrasonic detection circuit of FIG. 2 at this stage is shown with reference to Panel a in FIG. 4.

In the acquisition stage t2, the acquisition control signal Close is at a high level, and the reset control signal Rest, the compensation control signal VC and the read control signal Gate are all at a low level. Therefore, the drive transistor T1, the second transistor T2, the fourth transistor T4 and the fifth transistor T5 are closed, and the third transistor T3 is open. A signal received by the ultrasonic sensor Q is written into the first node N1. At the same time, due to the bootstrap effect of the first capacitor C1, the voltage of the second node N2 changes with the voltage of the first node N1. The equivalent circuit of the ultrasonic detection circuit of FIG. 2 at this stage is shown graphically with reference to Panel b in FIG. 4.

In the read stage t3, the compensation control signal VC and the read control signal Gate are both at a high level, the reset control signal Rest and the acquisition control signal Close are at low level. Therefore, the second transistor T2 and the third transistor T3 are turned off, and the drive transistor T1, the fourth transistor T4 and the fifth transistor T5 are turned on. The initial voltage of the output end Opt is set to 0V. The fourth transistor T4, the drive transistor T1 and the fifth transistor T5 form a path and the second node N2 voltage jumps. Due to the bootstrap effect of the first capacitor C1, the voltage of the first node N1 changes with the voltage of the second node N2. The output end Opt outputs a detection signal according to the potentials of the first node N1 and the second node N2. An equivalent circuit at this stage of the ultrasonic detection circuit shown in FIG. 2 is shown with reference to Panel c in FIG. 4.

Since in the reset compensation stage t1, the second node N2 is written into the threshold voltage Vth of the drive transistor T1. Since the first node N1 and the second node N2 are respectively connected to two plates of the first capacitor C1, and the bootstrap effect of the first capacitor C1. In the subsequent acquisition stage t2 and reading stage t3, the voltage difference between the first node N1 and the second node N2 always contains the threshold voltage Vth of the drive transistor T1, and substituting same into the Ids formula can eliminate the Vth, so that the influence of the threshold voltage Vth difference can be eliminated, the error of ultrasonic signal detection can be reduced, and the detection accuracy can be improved.

With reference to the signal timing diagrams shown in FIG. 3 and FIG. 9, the operation process of the ultrasonic detection circuit is described by taking the case where the drive transistors T1 to the fifth transistor T5 are all N-type transistors.

In a reset compensation stage t1, the reset control signal Rest, the acquisition control signal Close and the compensation control signal VC are all high-level, and the read control signal Gate is low-level. Therefore, the fifth transistor T5 is turned off, the drive transistor T1, the second transistor T2, the third transistor T3 and the fourth transistor T4 are turned on, and the reset signal Vbias is charged into the first node N1, so that the voltage of the first node N1 is Vbias. After the voltage of the second node N2 reaches Vbias−Vth, the drive transistor T1 is turned off, thereby achieving that the threshold voltage Vth of the drive transistor T1 is written to the second node N2, which serves to compensate for the difference in the threshold voltage Vth during the read stage t3. The equivalent circuit of the ultrasonic detection circuit of FIG. 3 at this stage is shown with reference to Panel a in FIG. 5.

In the acquisition stage t2, the acquisition control signal Close is at a high level, and the reset control signal Rest, the compensation control signal VC and the read control signal Gate are all at a low level. Therefore, the drive transistor T1, the second transistor T2, the fourth transistor T4 and the fifth transistor T5 are closed, and the third transistor T3 is open. A signal received by the ultrasonic sensor Q is written into the first node N1. At the same time, due to the bootstrap effect of the first capacitor C1, the voltage of the second node N2 changes with the voltage of the first node N1. The equivalent circuit of the ultrasonic detection circuit of FIG. 3 at this stage is shown graphically with reference to FIG. 5b.

In the read stage t3, the compensation control signal VC and the read control signal Gate are both at a high level, the reset control signal Rest and the acquisition control signal Close are at low level. Therefore, the second transistor T2 and the third transistor T3 are turned off, and the drive transistor T1, the fourth transistor T4 and the fifth transistor T5 are turned on. The initial voltage of the output end Opt is set to 0 V. The fourth transistor T4, the drive transistor T1 and the fifth transistor T5 form a path and the second node N2 voltage jumps. Due to the bootstrap effect of the first capacitor C1, the voltage of the first node N1 changes with the voltage of the second node N2. The output end Opt outputs a detection signal according to the potentials of the first node N1 and the second node N2. An equivalent circuit at this stage of the ultrasonic detection circuit shown in FIG. 3 is shown with reference to the Panel c in FIG. 5.

Since in the reset compensation stage t1, the second node N2 is written into the threshold voltage Vth of the drive transistor T1. Since the first node N1 and the second node N2 are respectively connected to two plates of the first capacitor C1, and the bootstrap effect of the first capacitor C1. In the subsequent acquisition stage t2 and reading stage t3, the voltage difference between the first node N1 and the second node N2 always contains the threshold voltage Vth of the drive transistor T1, and substituting same into the Ids formula can eliminate the Vth, so that the influence of the threshold voltage Vth difference can be eliminated, the error of ultrasonic signal detection can be reduced, and the detection accuracy can be improved.

The present disclosure provides an ultrasonic detection device including an underlayer substrate, a plurality of detection units located on one side of the underlayer substrate and including an ultrasonic sensor Q for receiving an ultrasonic signal reflected back and converting the received ultrasonic signal into an electrical signal, and an ultrasonic detection circuit as provided in any embodiment.

It will be appreciated by those skilled in the art that the ultrasonic detection device provided by the present disclosure has the advantages of the ultrasonic detection circuit described above. The ultrasonic devices provided by the present disclosure may be integrated into products such as ultrasonic imaging devices and fingerprint recognition devices.

The ultrasonic sensor Q receives the reflected ultrasonic signal and converts the received ultrasonic signal into an electrical signal, for example, in the range of 0V to 2V.

In some embodiments, the plurality of detection units are arranged in an array along a row direction and/or a column direction.

In some embodiments, the ultrasonic sensor Q is further configured to emit an ultrasonic signal in response to the first driving signal.

In particular implementations, a first driving signal may be provided to the ultrasonic sensor Q to cause the ultrasonic sensor Q to emit an ultrasonic signal after the last acquisition stage t2 and before the next acquisition stage t2.

In the present embodiment, the ultrasonic sensor Q performs the transmission and reception of the ultrasonic signal in time intervals, and the structure of the ultrasonic detection device can be simplified without a separate ultrasonic emitter.

In some embodiments, the ultrasonic detection device further includes an ultrasonic emitter for transmitting an ultrasonic signal in response to the second driving signal.

In the present embodiment, different devices are respectively used for the transmission and reception of the ultrasonic signal, i.e., an ultrasonic emitter for transmitting the ultrasonic signal and an ultrasonic sensor Q for receiving the ultrasonic signal, which is advantageous in improving detection efficiency and accuracy.

The present disclosure provides a driving method applied to an ultrasonic detection circuit as provided in any of the embodiments, as shown in FIG. 2 to FIG. 9, the driving method including.

In the reset compensation stage t1, a reset control signal is provided to the reset control end Rest, and a compensation control signal is provided to the compensation control end VC to conduct the reset module 21 and the compensation module 24. The reset signal is written into the first node N1, and the threshold voltage Vth of the drive transistor T1 is written into the second node N2.

In the acquisition stage t2, an acquisition control signal is provided to the acquisition control end Close to conduct the acquisition module 22 and write the signal received by the ultrasonic sensor Q into the first node N1.

In the read stage t3, a compensation control signal is provided to the compensation control end VC, and a read control signal is provided to the read control end Gate to conduct the compensation module 24 and the reading module 25. A detection signal is output at the output end Opt according to the potentials of the first node N1 and the second node N2.

In some embodiments, when the reset module 21 is connected to the first node N1 via the acquisition module 22, the reset compensation stage t1 further includes:
an acquisition control signal is provided to an acquisition control end Close to enable the acquisition module 22 to be conductive, and a reset signal is written into the first node N1 via the reset module 21 and the acquisition module 22 in sequence.

In some embodiments, when the ultrasonic detection circuit further includes a control signal generation module 61, and the control signal generation module 61 is respectively connected to a reset control end Rest, a read control end Gate and a compensation control end VC, the step of providing a compensation control signal to the compensation control end VC includes:
supplying the reset control signal to the reset control end Rest and the read control signal to the read control end Gate for generating the compensation control signal at the compensation control end VC.

In some embodiments, after the last acquisition stage t2 and before the next acquisition stage t2, it further includes the step below.

In the transmitting stage, a first driving signal is supplied to the ultrasonic sensor Q so that the ultrasonic sensor Q emits an ultrasonic signal.

It should be noted that the driving method may include further steps, which may be determined according to actual requirements, and the present disclosure is not limited thereto. With regard to the detailed description and technical effect of the driving method, reference is made to the above description of the embodiment of the ultrasonic detection circuit, which will not be repeated here.

In the present disclosure, the meaning of “multiple” refers to two or more, and the meaning of “at least one” refers to one or more, unless otherwise specified.

In the present disclosure, the terms “up”, “down”, etc. indicate orientation or positional relationships based on the orientation or positional relationships shown in the appended drawings, only for the convenience of describing the present disclosure and simplifying the description, and do not indicate or imply that the device or component referred to must have a specific orientation, be constructed and operated in a specific orientation, and therefore cannot be understood as a limitation of the present disclosure.

In the present disclosure, the terms “including”, “containing”, or any other variation thereof are intended to encompass non exclusive inclusion, such that a process, method, product, or equipment that includes a series of elements includes not only those elements, but also other elements not explicitly listed, or elements inherent to such process, method, product, or equipment. Without further limitations, the element defined by the statement ‘including one . . . ’ does not exclude the existence of other identical elements in the process, method, product, or device that includes the element in question.

The terms ‘one embodiment’, ‘some embodiments’, ‘exemplary embodiments’, ‘one or more embodiments’, ‘examples’, ‘one example’, ‘some examples’, etc. referred to in the present disclosure are intended to indicate that specific features, structures, materials, or characteristics related to the embodiment or example are included in at least one embodiment or example disclosed herein. The schematic representation of the above terms does not necessarily refer to the same embodiment or example. In addition, the specific features, structures, materials, or characteristics described may be included in any appropriate manner in any one or more embodiments or examples.

In the present disclosure, relational terms such as first and second are only used to distinguish one entity or operation from another, and do not necessarily require or imply any actual relationship or order between these entities or operations.

When describing some embodiments, expressions such as “coupling” and “connection” may be used. For example, in describing some embodiments, the term “connection” may be used to indicate that two or more components have direct physical or electrical contact with each other. For example, in describing some embodiments, the term “coupling” may be used to indicate that two or more components have direct physical or electrical contact. However, the term “coupled” or “communicably coupled” may also refer to two or more components that do not have direct contact with each other but still cooperate or interact with each other. The embodiments disclosed here are not necessarily limited to the content of the present disclosure.

At least one of A, B, and C “has the same meaning as” at least one of A, B, or C “and includes the following combinations of A, B, and C: only A, only B, only C, combinations of A and B, combinations of A and C, combinations of B and C, and combinations of A, B, and C.

A and/or B “includes the following three combinations: only A, only B, and a combination of A and B.

As used in the present disclosure, the term ‘if’ is optionally interpreted as meaning ‘when’ or ‘at’ or ‘in response to a determination’ or ‘in response to a detection’ depending on the context. Similarly, depending on the context, the phrases “if determined . . . ” or “if [stated condition or event] is detected” can be interpreted as referring to “when determined . . . ” or “in response to determining . . . ” or “when [stated condition or event] is detected” or “in response to detecting [stated condition or event]”.

The use of “for” or “configured to” in the present disclosure implies an open and inclusive language, which does not exclude devices that are applicable or configured to perform additional tasks or steps.

The use of “based on” or “according to” in the present disclosure implies openness and inclusiveness. A process, step, calculation, or other action based on one or more of the conditions or values described, which may be based on other conditions or beyond the values described in practice. The process, steps, calculations, or other actions based on one or more of the stated conditions or values may, in practice, be based on other conditions or beyond the stated values.

As used in the present disclosure, “about”, “roughly”, or “approximately” include the values described and the average value within an acceptable deviation range of a specific value, where the acceptable deviation range is determined by those skilled in the art taking into account the measurement being discussed and the errors associated with the measurement of a specific quantity (i.e., limitations of the measurement system).

As used in the present disclosure, “parallel”, “vertical”, “equal”, and “flush” include the situations described and situations that are similar to the described situations, and the range of the similar situations is within an acceptable deviation range, where the acceptable deviation range is determined by those skilled in the art considering the measurement being discussed and the errors associated with the measurement of a specific quantity (i.e., the limitations of the measurement system). For example, “parallelism” includes absolute parallelism and approximate parallelism, where the acceptable deviation range for approximate parallelism can be within 5 degrees of deviation; Vertical “includes absolute vertical and approximate vertical, where the acceptable deviation range for approximate vertical can also be within 5°, for example. ‘Equality’ includes absolute equality and approximate equality, where the acceptable deviation range for approximate equality can be, for example, equal. The difference between the two is less than or equal to 5% of either one. ‘Leveling’ includes absolute leveling and approximate leveling, where the acceptable deviation range for approximate leveling can be, for example, that the distance between two levels of leveling is less than or equal to 5% of either dimension.

It should be understood that when a layer or component is referred to as being on another layer or substrate, it may be directly on another layer or substrate, or there may be an intermediate layer between the layer or component and another layer or substrate.

the present disclosure describes exemplary embodiments with reference to cross-sectional and/or plan views as idealized illustrative figures. In the attached figure, the thickness of the layers and regions has been enlarged for clarity. Therefore, it can be assumed that there may be changes in the shape relative to the drawings due to factors such as manufacturing technology and/or tolerances. Therefore, the exemplary embodiments should not be interpreted as limited to the shapes of the regions shown in the present disclosure, but rather include shape deviations caused by, for example, manufacturing. For example, etched areas shown as rectangles typically have curved features. Therefore, the areas shown in the figures are essentially illustrative, and their shapes are not intended to show the actual shape of the device's area, nor are they intended to limit the scope of the exemplary embodiments.

Finally, it should be noted that the above embodiments are only used to illustrate the disclosed technical solution and not to limit it; Although the present disclosure has been described in detail with reference to the aforementioned embodiments, those skilled in the art should understand that they can still modify the technical solutions described in the aforementioned embodiments, or equivalently replace some of the technical features; And these modifications or substitutions do not depart from the essence and scope of the corresponding technical solutions disclosed in the present disclosure.

1. An ultrasonic detection circuit, comprising a reset module, an acquisition module, a drive transistor, a storage module, a compensation module and a reading module;
wherein the reset module is respectively connected to a reset control end, a reset signal end and a first node, and is configured to write a reset signal of the reset signal end into the first node in response to a reset control signal of the reset control end;
the acquisition module is connected to an acquisition control end, an ultrasonic sensor and the first node, and is configured to write a signal received by the ultrasonic sensor into the first node in response to an acquisition control signal of the acquisition control end;
the drive transistor and a control electrode are connected to the first node, a first electrode is connected to the compensation module, and a second electrode is connected to the second node;
the storage module is respectively connected to the first node and the second node, and is configured to store a voltage between the first node and the second node;
the compensation module is further connected to a compensation control end and a first voltage end, and is configured to write a threshold voltage of the drive transistor into the second node in response to a compensation control signal of the compensation control end; and
the reading module is respectively connected to a read control end, the second node and an output end, and is configured to output a detection signal at the output end according to potentials of the first node and the second node in response to a reading control signal of the read control end.

2. The ultrasonic detection circuit according to claim 1, wherein the reset module is directly connected to the first node; or
the reset module is connected to the first node via the acquisition module, and the reset module is specifically configured to write the reset signal into the acquisition module in response to the reset control signal; and the acquisition module is also configured to write the reset signal into the first node in response to the acquisition control signal.

3. The ultrasonic detection circuit according to claim 2, wherein the reset module comprises:
a second transistor, wherein a control electrode is connected to the reset control end, a first electrode is connected to the reset signal end, and a second electrode is connected to the first node directly or via the acquisition module.

4. The ultrasonic detection circuit according to claim 2, wherein the acquisition module comprises:
a third transistor, wherein a control electrode is connected to the acquisition control end, a first electrode is connected to the ultrasonic sensor, or the first electrode is respectively connected to the ultrasonic sensor and the reset module, and a second electrode is connected to the first node.

5. The ultrasonic detection circuit according to claim 1, wherein the storage module comprises:
a first capacitor, wherein a first electrode is connected to the first node, and a second electrode is connected to the second node.

6. The ultrasonic detection circuit according to claim 1, wherein the compensation module comprises:
a fourth transistor, wherein a control electrode is connected to the compensation control end, a first electrode is connected to the first voltage end, and a second electrode is connected to a first electrode of the drive transistor.

7. The ultrasonic detection circuit according to claim 1, wherein the reading module comprises:
a fifth transistor, wherein a control electrode is connected to the read control end, a first electrode is connected to the second node, and a second electrode is connected to the output end.

8. The ultrasonic detection circuit according to claim 1, wherein the ultrasonic detection circuit further comprises:
a control signal generation module respectively connected to the reset control end, the read control end and the compensation control end and configured to generate the compensation control signal at the compensation control end in response to the reset control signal and the read control signal.

9. The ultrasonic detection circuit according to claim 8, wherein the control signal generation module comprises:
a sixth transistor, wherein a control electrode and a first electrode are all connected to the reset control end, and a second electrode is connected to the compensation control end; and
a seventh transistor, wherein a control electrode and a first electrode are all connected to the read control end, and a second electrode is connected to the compensation control end.

10. An ultrasonic detection device, comprising:
an underlayer substrate;
a plurality of detection units located on a side of the underlayer substrate, wherein the detection units comprises an ultrasonic sensor, and the ultrasonic detection circuit according to claim 1, and the ultrasonic sensor is configured to receive an ultrasonic signal reflected back and convert the received ultrasonic signal into an electrical signal.

11. The ultrasonic detection device according to claim 10, wherein the plurality of detection units are arrayed in a row direction and/or a column direction.

12. The ultrasonic detection device according to claim 10, wherein the ultrasonic sensor is further configured to emit an ultrasonic signal in response to a first driving signal; or
the ultrasonic detection device further comprises an ultrasonic emitter, and the ultrasonic emitter is configured to emit an ultrasonic signal in response to a second driving signal.

13. A driving method applied to the ultrasonic detection circuit according to claim 1, wherein the driving method comprises:
a reset compensation stage, providing a reset control signal to the reset control end and providing a compensation control signal to the compensation control end, so that the reset module and the compensation module are conductive, writing the reset signal into the first node, and writing the threshold voltage of the drive transistor into the second node;
an acquisition stage, providing an acquisition control signal to the acquisition control end to enable the acquisition module to conduct, and writing a signal received by the ultrasonic sensor into the first node; and
a reading stage, providing a compensation control signal to the compensation control end and providing a reading control signal to the read control end, so that the compensation module and the reading module are conductive, and outputting a detection signal at the output end according to the potentials of the first node and the second node.

14. The driving method according to claim 13, wherein when the reset module is connected to the first node by the acquisition module, the reset compensation stage further comprises:
providing an acquisition control signal to the acquisition control end to conduct the acquisition module, wherein the reset signal is written into the first node via the reset module and the acquisition module in sequence.

15. The driving method according to claim 13, wherein when the ultrasonic detection circuit further comprises a control signal generation module, and the control signal generation module is respectively connected to the reset control end, the read control end and the compensation control end, a step of providing a compensation control signal to the compensation control end comprises:
providing a reset control signal to the reset control end and providing a read control signal to the read control end to generate the compensation control signal at the compensation control end.

16. The driving method according to claim 1, wherein after a previous acquisition stage and before a next acquisition stage, the driving method further comprises:
an emitting stage, providing a first driving signal to the ultrasonic sensor to cause the ultrasonic sensor to emit an ultrasonic signal.
